(19) United States
(12) Patent Application Publication
(10) Pub. No.: US 2019/0239565 A1
(43) Pub. Date: Aug. 8, 2019

(54) BATTERY ASSEMBLY FOR ELECTRONIC CIGARETTE AND ELECTRONIC CIGARETTE

(71) Applicant: Changzhou Patent Electronic Technology Co., LTD, Changzhou (CN)

(72) Inventors: Weihua QIU, Changzhou (CN); Wenbang WANG, Changzhou (CN)

(21) Appl. No.: 16/259,569

(22) Filed: Jan. 28, 2019

(30) Foreign Application Priority Data
Feb. 2, 2018 (CN) 201820191073.7

(51) Int. Cl. A24F 47/00; A61M 11/04; H02J 7/00; H01M 10/44; H01M 2/10; H01H 13/14

(52) U.S. Cl. CPC A24F 47/008; A61M 11/042; H02J 7/0052; H01M 2010/4271; H01M 2/1055; H01H 13/14; H01M 10/44

(57) ABSTRACT

A battery assembly for electrical cigarette includes a housing provided with a cavity for receiving a battery, a main button movably disposed on the housing and a locking member slidably located on the housing, sliding the locking member can lock the main button such that the main button is not triggered. The battery assembly can effectively reduce the battery depletion. An electronic cigarette is also provided.

### FIELD

The present disclosure relates to the technical field of electronic cigarette, and in particular to a battery assembly for an electronic cigarette and an electronic cigarette using the same.

### BACKGROUND

Electronic cigarettes, also known as virtual cigarettes, electronic nebulizers, electronic cigars, have the same physical appearance and similar taste as conventional cigarettes, and are mainly used to simulate smoking.

Currently, an electronic cigarette includes a battery assembly that provides a power supply function and an atomizer assembly that provides an atomizing function. A unit such as a main button switch is often disposed on casing of the battery assembly. Pressing the main button switch will trigger the battery assembly. However, during the period when not using electronic cigarettes, the user often inadvertently touches the main button to trigger the battery, increasing the battery depletion and shortening the battery life.

### SUMMARY

The object of the present disclosure is to provide a battery assembly capable of reducing the battery depletion and an electronic cigarette having the battery assembly.

A battery assembly for electrical cigarette includes a housing provided with a cavity for receiving a battery, a main button movably disposed on the housing and a locking member slidably located on the housing, sliding the locking member can lock the main button such that the main button is not triggered.

In one embodiment, the main button defines a stopping groove, the opening of the stopping groove faces the locking member, one end of the locking member is formed with a locking portion, the locking portion can extend into the stopping groove to latch the main button.

In one embodiment, the housing is provided with a button hole and a sliding groove, the button hole extends through the housing, the sliding groove is recessed on the surface of the housing and is in communication with the button hole, the main button is movably disposed in the button hole, the locking member is slidably received in the sliding groove.

In one embodiment, the locking member includes an operating portion, the locking portion protrudes from the stopping groove, the operating portion is slidably disposed in the sliding groove.

In one embodiment, the sliding groove is further provided with a sliding slot, the sliding slot are respectively recessed on the two opposite sides of the sliding groove, sliding ribs are respectively protruded from the opposite sides of the locking member, the sliding ribs are slidably received in the sliding groove.

In one embodiment, the battery assembly further includes elastic member, the bottom of the locking member is recessed with a receiving groove, the elastic member is received in the receiving groove and slidably abuts against the underside of the sliding groove.

In one embodiment, the main button includes a pressing body, a pressing portion protruding from the pressing body, a stopping groove is recessed inwardly from the peripheral wall of the pressing body facing the locking member.

In one embodiment, the battery assembly further includes a mounting ring is disposed in the button hole, the mounting ring is sleeved outside of the main button, one side of the mounting ring is provided with a notch, the notch is in communication with the stopping groove.

In one embodiment, the mounting ring a sleeve portion and a limiting portion protruding from the sleeve portion, the sleeve portion is sleeved on the periphery of the main button, the notch is defined at the sleeve portion, the limiting portion abuts against the surface of the housing.

In one embodiment, when the locking member moves away from the main button to make the locking portion passes through the notch and exits the stopping groove, then the main button is in the unlocked state.

In one embodiment, the main button includes a pressing body, a pressing portion protruding from the pressing body, and two sidewalls with blocking function, the sidewalls are respectively protruded from the two opposite sides of the pressing portion, the stopping groove is formed between two of the sidewalls, the opening of the stopping groove faces the locking member.

An electronic cigarette includes an atomizer, and any one of the above battery assembly, the atomizer is electrically connected to the battery assembly.

The beneficial effects of the device are:

Since the battery assembly for electronic cigarettes has a locking function, the user can slide the locking member relative to the sliding grooves to lock or unlock the battery assembly. When the locking member passes through the stopping groove, the main button cannot be pressed to activate the battery assembly. The battery assembly is activated or deactivated by sliding the lock element. When the user does not use the electronic cigarette, the locking member can be moved towards to the main button, so that the main button cannot be pressed. Therefore, even if the main button is inadvertently touched during non-operation, the battery assembly cannot be triggered to operate, thereby, the battery depletion of the battery assembly is better reduced, and the service life is prolonged. The locking member abuts against the housing through the elastic member, thereby enhancing the hand feeling when the locking member slides. The battery assembly has a convenient structure and simple operation, and can better protect the safety of the battery assembly.

### BRIEF DESCRIPTION OF THE DRAWINGS

FIG. 1 shows the states when the main button of an electronic cigarette of the disclosure is turned on and turned off;

FIG. 2 shows the states when the locking member of the electronic cigarette of FIG. 1 is open or closed;

FIG. 3 is a perspective structural view of a battery assembly for the electronic cigarette of FIG. 2;

FIG. 4 is a perspective view of the lock member of the electronic cigarette of FIG. 3;

FIG. 5 is a perspective view of the main button of the electronic cigarette of FIG. 4.

The following table list various components and reference numerals thereof.

| | |
|---|---|
| Battery assembly 30 | Main button 32 |
| Elastic member 34 | Button hole 311 |
| Sliding slot 3121 | Pressing portion 322 |
| Stopping groove 324 | Operating portion 332 |
| Receiving groove 334 | Locking member 33 |
| Housing 31 | Sliding groove 312 |
| Mounting ring 35 | Sidewall 323 |
| Pressing body 321 | Sliding rib 333 |
| Locking portion 331 | Notch 351 |

### DETAILED DESCRIPTION OF PREFERRED EMBODIMENTS

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

Several definitions that apply throughout this disclosure will now be presented.

The term “coupled” is defined as connected, whether directly or indirectly through intervening components, and is not necessarily limited to physical connections. The connection can be such that the objects are permanently connected or releasably connected. The term “comprising,” when utilized, means “including, but not necessarily limited to”; it specifically indicates open-ended inclusion or membership in the so-described combination, group, series and the like.

When a feature or element is herein referred to as being “on” another feature or element, it can be directly on the other feature or element or intervening features and/or elements may also be present.

Terminology used herein is for the purpose of describing particular embodiments only and is not intended to be limiting of the disclosure. As used herein, the term “and/or” includes any and all combinations of one or more of the associated listed items and may be abbreviated as “/”.

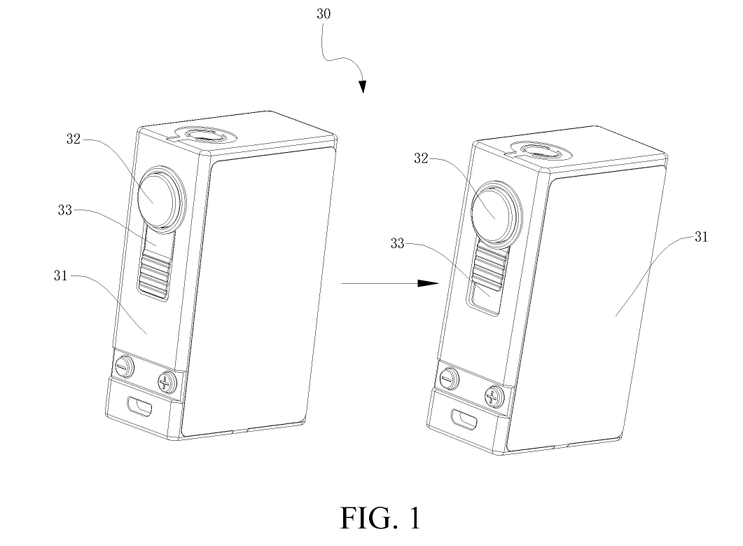

Referring to FIGS. 1-3, an embodiment of the present disclosure provides an electronic cigarette including an atomizer (not shown), a mouthpiece (not shown), and a battery assembly 30. The atomizer is electrically connected to the battery assembly 30, the mouthpiece is disposed on the atomizer.

The battery assembly 30 includes a housing 31, a battery (not shown), a main button 32, a locking member 33, an elastic member 34, and a mounting ring 35. The housing 31 is provided with a cavity for receiving a battery, the battery is received in the housing 31. The main button 32 is movably disposed on the housing 31. The locking member 33 is slidably located on the housing 31 and is adjacent to the main button 32. The locking member 33 is configured for sliding to lock the main button 32, so that the main button 32 cannot be triggered. In the embodiment, the locking member 33 is latched by the main button 32. In other embodiments, the main button 32 is magnetically locked by the locking member 33, or the main button is threaded locked by the locking member 33. The elastic member 34 is disposed on the locking member 33, the mounting ring 35 is sleeved on the main button 32.

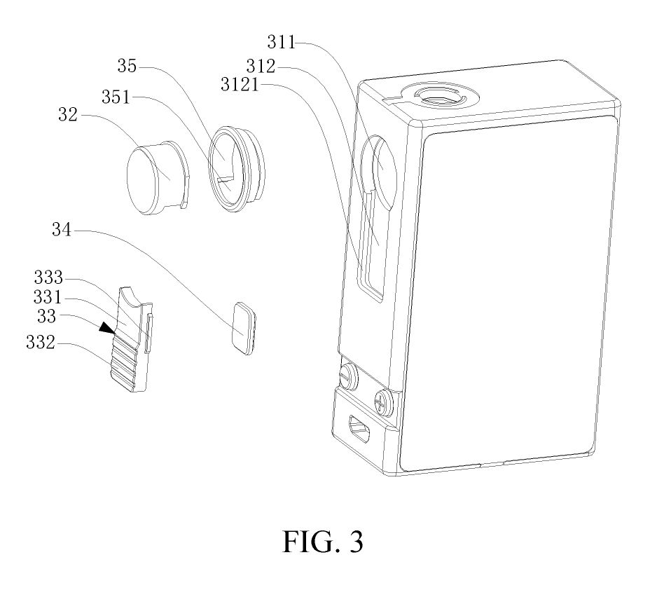

Referring to FIG. 3, the housing 31 is provided with a button hole 311 and a sliding groove 312. The button hole 311 extends through the housing 31. The sliding groove 312 is recessed on the surface of the housing 31 and is in communication with the button hole 311. Further, the sliding groove 312 is further provided with two sliding slots 3121. Specifically, the sliding slots 3121 are respectively recessed on the two opposite sides of the sliding groove 312. In this embodiment, the bottom of the sliding groove 312 is provided with a bottom plate (not shown), the elastic member 34 elastically resist the bottom plate, so as to fit the locking member 33 into the sliding groove 312 more tightly. It is understood that, in other embodiments, the bottom plate may not be provided. It can be understood that, the elastic member can be a silicone pad, a rubber pad, but is not limited to the above materials.

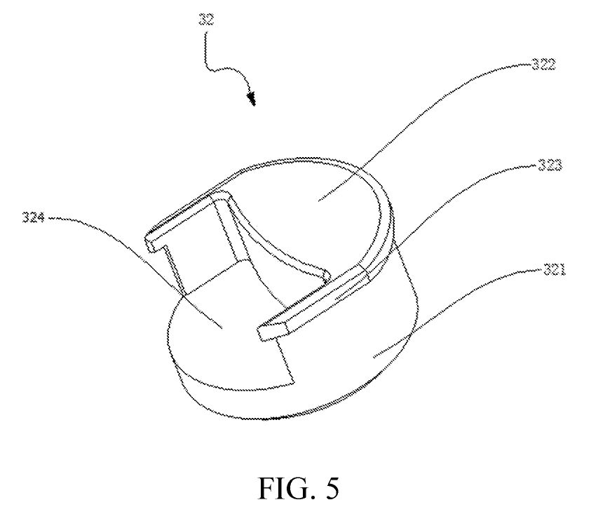

Referring to FIG. 3 and FIG. 5, the main button 32 is movably disposed in the button hole 311. The main button 32 includes a pressing body 321, a pressing portion 322 protruding from the pressing body 321, and two sidewalls 323 with blocking function. The sidewalls 323 are respectively protruded from the two opposite sides of the pressing portion 322. A side of the pressing body 321 facing the locking member 33 is recessed inward to form a stopping groove 324. The stopping groove 324 is formed between the two sidewalls 323. The opening of the stopping groove 324 faces the locking member 33. It should be noted that, the main button 32 of the present disclosure is a type of button switch, which differs from the conventional button switch only in that the pressed member (pressing body 321) has a different structure, and the structure of other portions is not limited.

In an embodiment not shown, the main button 32 includes a return spring, a stationary contact, and a movable contact, in addition to the pressing body 321. The movable contact is mounted on the lower end of the pressing body 321, the stationary contact is fixedly mounted inside the housing 31. The return spring is disposed along the operating direction of the pressing body 321, one end of the return spring abuts the pressing body 321, so that when the pressing body 321 is not pressed, the movable contact is separated from the stationary contact by the elastic force of the return spring. Further, the stationary contact and the movable contact can be made of metal reed or conductive rubber.

Furthermore, operating the pressing body 321 can turn on or off the connection between the movable contact and the stationary contact, thereby implementing the circuit to be turned on or off. When the pressing body 321 is not pressed, the movable contact is disconnected from the stationary contact, the pressing button 321 is not triggered. Pressing the pressing body 321, the movable contact is connected with the stationary contact, the main button 32 is triggered. When the pressing body 321 is released, the original working state of the pressing body 321 is restored under the action of the return spring, that is, the pressing body 321 is restored to the original position, the movable contact and the stationary contact are separated again.

When the pressing body 321 is pressed, the pressing portion 322 moves in the operational direction of the main button 32 such that other switching members (such as moving contact and stationary contact) come into contact with each other, thereby the main button 32 is triggered. In the embodiment, the pressing body 321 defines a stopping groove 324 corresponding to the locking member 33. Specifically, the stopping groove 324 is below the upper end surface of the pressing body 321 and is recessed inwardly from the peripheral wall of the pressing body 321 facing the locking member 33. The stopping groove 324 is surrounded by the pressing body 321, the pressing portion 322, and the two sidewalls 323. The stopping groove 324 is provided with an opening that extends through the bottom surface thereof and also extends through the sidewall thereof facing locking member. In other embodiments, the structure of the stopping groove 324 is not limited, only after the locking member 33 inserted into the stopping groove 324, the main button 32 can interfere with the locking member 33 along the stroke direction of the main button 32. In the embodiment, the main button 32 is made of one or more of materials such as silicone rubber or rubber, but is not limited to the above materials. A peripheral edge (not shown) is protruded from the bottom of the main button 32. The outer diameter of the peripheral edge is larger than the diameter of the button hole 311, the main button 32 is inserted into the button hole 311 through the periphery edge to prevent the main button 32 from being separate from the button hole 311.

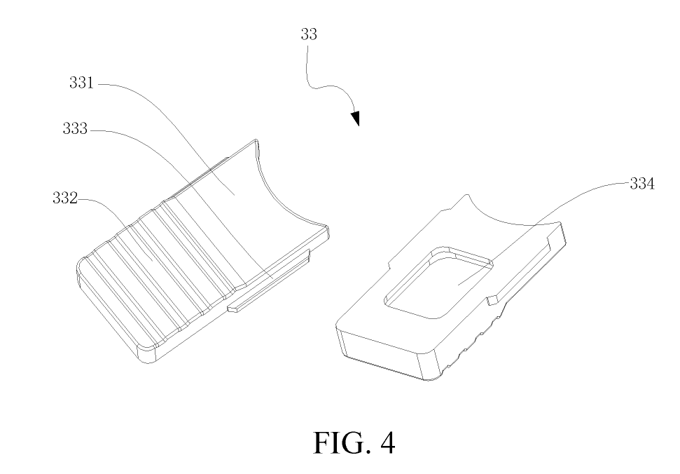

Referring to FIG. 4, the locking member 33 is slidably received in the sliding groove 312. The locking member 33 includes a locking portion 331, an operating portion 332 and a sliding rib 333. The locking portion 331 is protruded from one end of the operating portion 322, the locking portion 331 can extend into the stopping groove 324 to latch the main button 32. The operating portion 332 is slidably disposed in the sliding groove 312. In this embodiment, the sliding ribs 333 are two, the two sliding ribs 333 are respectively protruded from the opposite sides of the locking member 33 and extend to the operating portion 332. The sliding ribs 333 are slidably disposed in the two sliding slots 3121, respectively. Further, the end surface of the locking portion 331 which is in contact with the groove wall of the stopping groove 324 is adapted to the shape of the groove wall of the stopping groove 324. In the embodiment, the end surface of the locking portion 331 which is in contact with the groove wall of the stopping groove 324 is a curved surface. In other embodiments, it may be a flat surface. Furthermore, the bottom of the locking member 33 is recessed with a receiving groove 334, the receiving groove 334 can be used for mounting the elastic member. When the main button 32 is in the locked state, the locking portion 331 protrudes from the stopping groove 324 to latch the main button 32, the elastic member 34 is received in the receiving groove 334 and slidably abuts against the underside of the sliding groove 312.

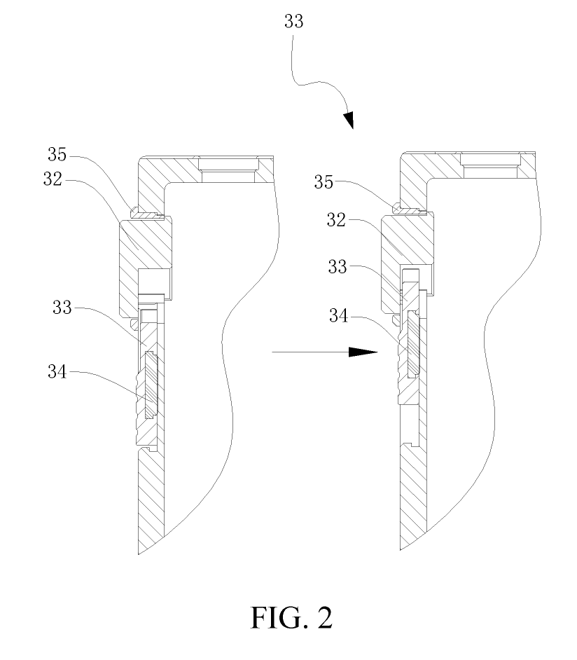

Referring to FIGS. 2-3, the mounting ring 35 is disposed in the button hole 311, the mounting ring 35 is sleeved outside of the main button 32. The mounting ring 35 includes a sleeve portion (not shown) and a limiting portion (not shown) protruding from the sleeve portion. The sleeve portion is sleeved on the periphery of the main button 32. One side of the mounting ring 35 is provided with a notch 351. The notch 351 is in communication with the stopping groove 324. The notch 351 is defined at the sleeve portion, the limiting portion abuts against the surface of the housing 31. In the embodiment, the limiting portion is an annular abutting edge. In the embodiment, the mounting ring 35 is made of one or more of hard materials such as aluminum, iron, copper, and plastic, but is not limited to the above materials. The mounting ring 35 has a good limit fixing function for the main button 32.

During assembly, the mounting ring 35 is disposed in the button hole 311, the main button 32 is disposed in the mounting ring 35, the notch 351 is in communication with the stopping groove 324, so that the locking member 33 is locked in the notch 351 and the stopping groove 324.

The use, when the user does not use the electronic cigarette, the locking member 33 can be moved toward the main button 32, and the locking portion 331 of the locking member 33 passes through the notch 351 and is latched into the stopping groove 324. At this time, the lower end surface of the pressing body 321 cannot be moved downward due to the resistance with the locking portion 331, so that the main button 32 cannot be pressed, the main button 32 is in the locked state. When the user needs to use the electronic cigarette, moving the locking member 33 away from the main button 32 to make the locking portion 331 of the locking member 33 passes through the notch 351 and exits the stopping groove 324, then the main button 32 is in the unlocked state.

Since the battery assembly 30 for electronic cigarettes has a locking function, the user can slide the locking member 33 relative to the sliding slots 3121 to lock or unlock the battery assembly 30. When the locking member 33 passes through the stopping groove 324, the main button cannot be pressed to activate the battery assembly 30. The battery assembly 30 is activated and closed by sliding the locking member 33. When the user does not use the electronic cigarette, the locking member 33 can be moved towards to the main button 32, so that the main button 32 cannot be pressed. Therefore, even if the main button 32 is inadvertently touched during non-operation, the battery assembly 30 cannot be triggered to operate, thereby, the depletion of the battery assembly 30 is better reduced, and the service life is prolonged. It should be noted that, when the main button 32 of the disclosure is triggered, the functions of the battery assembly 30 and/or the electronic cigarette include powering the atomizer (lighting), locking the screen, unlocking the screen, turning on, turning off, adjust parameters, mode selection, and so on. The elastic member 34 is received in the receiving groove 334 and slidably abuts against the bottom surface of the sliding groove 312. The locking member 33 abuts against the housing 31 through the elastic member 34, thereby enhancing the hand feeling when the locking member 33 slides. The battery assembly has a convenient structure and simple operation, and can better protect the safety of the battery assembly.

The above-mentioned embodiments merely represent several implementations of the present application, and the descriptions thereof are more specific and detailed, but they shall not be understood as a limitation on the scope of the present application. It should be noted that, for those of ordinary skill in the art, variations and improvements may still be made without departing from the concept of the present application, and all of which shall fall into the protection scope of the present application. Therefore, the scope of protection of the present application shall be subject to the appended claims.

What is claimed is:

1. A battery assembly for electrical cigarette, comprising:
a housing provided with a cavity for receiving a battery;
a main button movably disposed on the housing; and
a locking member slidably located on the housing, sliding the locking member locks the main button such that the main button is not triggered.

2. The battery assembly according to claim 1, wherein the main button defines a stopping groove, the opening of the stopping groove faces the locking member, one end of the locking member is formed with a locking portion, the locking portion can extend into the stopping groove to latch the main button.

3. The battery assembly according to claim 2, wherein the housing is provided with a button hole and a sliding groove, the button hole extends through the housing, the sliding groove is recessed on the surface of the housing and is in communication with the button hole, the main button is movably disposed in the button hole, the locking member is slidably received in the sliding groove.

4. The battery assembly according to claim 3, wherein the locking member comprises an operating portion, the locking portion protrudes from the stopping groove, the operating portion is slidably disposed in the sliding groove.

5. The battery assembly according to claim 4, wherein the sliding groove is further provided with a sliding slot, the sliding slot are respectively recessed on the two opposite sides of the sliding groove, sliding ribs are respectively protruded from the opposite sides of the locking member, the sliding ribs are slidably received in the sliding groove.

6. The battery assembly according to claim 3, wherein the battery assembly further comprises elastic member, the bottom of the locking member is recessed with a receiving groove, the elastic member is received in the receiving groove and slidably abuts against the underside of the sliding groove.

7. The battery assembly according to claim 2, wherein the main button comprises a pressing body, a pressing portion protruding from the pressing body, a stopping groove is recessed inwardly from the peripheral wall of the pressing body facing the locking member.

8. The battery assembly according to claim 3, wherein the battery assembly further comprises a mounting ring is disposed in the button hole, the mounting ring is sleeved outside of the main button, one side of the mounting ring is provided with a notch, the notch is in communication with the stopping groove.

9. The battery assembly according to claim 8, wherein the mounting ring a sleeve portion and a limiting portion protruding from the sleeve portion, the sleeve portion is sleeved on the periphery of the main button, the notch is defined at the sleeve portion, the limiting portion abuts against the surface of the housing.

10. The battery assembly according to claim 9, wherein when the locking member moves away from the main button to make the locking portion passes through the notch and exits the stopping groove, then the main button is in the unlocked state.

11. The battery assembly according to claim 2, wherein the main button comprises a pressing body, a pressing portion protruding from the pressing body, and two sidewalls with blocking function, the sidewalls are respectively protruded from the two opposite sides of the pressing portion, the stopping groove is formed between two of the sidewalls, the opening of the stopping groove faces the locking member.

12. An electronic cigarette, comprising:
an atomizer; and
a battery assembly electrically connected to the atomizer, the battery assembly comprises:
a housing provided with a cavity for receiving a battery;
a main button movably disposed on the housing; and
a locking member slidably located on the housing, sliding the locking member can lock the main button such that the main button is not triggered.

13. The electronic cigarette according to claim 12, wherein the main button defines a stopping groove, the opening of the stopping groove faces the locking member, one end of the locking member is formed with a locking portion, the locking portion can extend into the stopping groove to latch the main button.

14. The electronic cigarette according to claim 13, wherein the housing is provided with a button hole and a sliding groove, the button hole extends through the housing, the sliding groove is recessed on the surface of the housing and is in communication with the button hole, the main button is movably disposed in the button hole, the locking member is slidably received in the sliding groove.

15. The electronic cigarette according to claim 14, wherein the locking member comprises an operating portion, the locking portion protrudes from the stopping groove, the operating portion is slidably disposed in the sliding groove.

16. The electronic cigarette according to claim 15, wherein the sliding groove is further provided with a sliding slot, the sliding slot are respectively recessed on the two opposite sides of the sliding groove, sliding ribs are respectively protruded from the opposite sides of the locking member, the sliding ribs are slidably received in the sliding groove.

17. The electronic cigarette according to claim 14, wherein the battery assembly further comprises elastic member, the bottom of the locking member is recessed with a receiving groove, the elastic member is received in the receiving groove and slidably abuts against the underside of the sliding groove.

18. The electronic cigarette according to claim 13, wherein the main button comprises a pressing body, a pressing portion protruding from the pressing body, a stopping groove is recessed inwardly from the peripheral wall of the pressing body facing the locking member.

19. The electronic cigarette according to claim 14, wherein the battery assembly further comprises a mounting ring is disposed in the button hole, the mounting ring is sleeved outside of the main button, one side of the mounting ring is provided with a notch, the notch is in communication with the stopping groove.

20. The electronic cigarette according to claim 19, wherein the mounting ring a sleeve portion and a limiting portion protruding from the sleeve portion, the sleeve portion is sleeved on the periphery of the main button, the notch is defined at the sleeve portion, the limiting portion abuts against the surface of the housing.
